(19) United States
(12) Patent Application Publication
(10) Pub. No.: US 2024/0018482 A1
(43) Pub. Date: Jan. 18, 2024

(54) CRYOPRESERVED ENDOTHELIAL CELL COMPOSITIONS

(71) Applicant: Angiocrine Bioscience, Inc., San Diego, CA (US)

(72) Inventors: Michael Daniel Ginsberg, San Diego, CA (US); Lee Landeen, San Diego, CA (US); Paul William Finnegan, Del Mar, CA (US); John K Fraser, San Diego, CA (US)

(21) Appl. No.: 18/041,080

(22) PCT Filed: Aug. 10, 2021
(86) PCT No.: PCT/US21/45388

§ 371 Date: Feb. 9, 2023

Related U.S. Application Data
(60) Provisional application No. 63/063,668, filed on Aug. 10, 2020.

(51) Int. Cl. C12N 5/071; C12N 15/86; A01N 1/02

(52) U.S. Cl. CPC C12N 5/069; C12N 15/86; A01N 1/0221; A01N 1/0242; C12N 2502/1352; C12N 2501/998

(57) ABSTRACT

The present invention provides compositions comprising high densities of endothelial cells (such as human umbilical vein endothelial cells) in freezing media, methods of producing such compositions, and methods of using such compositions in the preparation of therapeutic endothelial cell compositions. Such compositions and methods provide numerous advantages including eliminating the requirement to remove cryopreservatives before administration of therapeutic endothelial cell compositions to human subjects and requiring minimal manipulations and human interventions before use in therapeutic methods.

### CROSS-REFERENCE TO RELATED APPLICATIONS

This application claims the benefit of priority of U.S. Provisional Patent Application No. 63/063,668 filed on Aug. 10, 2020, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 10, 2021, is named Angiocrine_027_WO1_SL.txt and is 1,543 bytes in size.

### INCORPORATION BY REFERENCE

For the purposes of only those jurisdictions that permit incorporation by reference, the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Many of the general teachings provided in U.S. Pat. No. 8,465,732 can be used in conjunction with the present invention, or can be adapted for use with the present invention. Accordingly, the entire contents of U.S. Pat. No. 8,465,732 are hereby expressly incorporated by reference into the present application.

### BACKGROUND

Endothelial cells (ECs), such as human umbilical vein endothelial cells (HUVECs), are widely used in research and are also in clinical development for cell therapy applications. ECs (such as HUVECs) are typically frozen at a concentration of about 1 million cells per ml in a cell freezing medium containing one or more cryopreservatives such as dimethyl sulfoxide (DMSO) (see, for example, Polchow et al., (2012), “Cryopreservation of human vascular umbilical cord cells under good manufacturing practice conditions for future cell banks,” Journal of Translational Medicine, 10:98; Sultani et al., (2016), “Improved Cryopreservation of Human Umbilical Vein Endothelial Cells: A Systematic Approach,” Sci. Rep., 6, 34393; and Pegg et al., (2002), “Cryopreservation of vascular endothelial cells as isolated cells and as monolayers.” Cryobiology, 44, 46-53). For clinical applications vials of ECs are provided to users frozen and then thawed, pelleted to separate the ECs from the freezing medium and cryopreservatives, resuspended in a physiological saline, and transferred to an infusion bag before they can be administered to a patient. The contents of several vials of frozen ECs may need to be combined and/or the ECs need to be expanded in culture to have sufficient cells for administration to a patient. The entire process from receipt of the frozen cells to administration of the cells to patients involves numerous manipulations including multiple centrifugation steps, container changes, and media/solution changes—each of which carries a risk of contamination. The process is also very labor and time intensive and susceptible to human error. As such, there is a need in the art for the provision of compositions and methods that could allow the entire process—from receipt of frozen ECs to use of the ECs (for example by infusion of the cells into a patient)—to be performed safely and efficiently with minimal manipulation. The present invention addresses this need.

### SUMMARY OF THE INVENTION

The present invention is based, in part, upon the discovery that ECs, such as HUVECs, can be frozen and thawed at densities of about 100 million cells per ml with excellent cell recovery and cell viability, and that the thawed cells can then be simply diluted, with or without removal of cryopreservatives, to generate a therapeutic composition that can be safely administered to human patients. To the best of our knowledge, freezing and thawing ECs at such a density is unprecedented.

Typically, ECs are frozen and thawed at concentrations about 20-fold to 100-fold lower than the concentrations that we describe herein. For example, a publication by Sultani et al. entitled “Improved Cryopreservation of Human Umbilical Vein Endothelial Cells: A Systematic Approach” described work to develop an improved HUVEC cryopreservation system. See Sultani et al., 2016, Sci. Rep., Vol. 6, 34393, p. 1-14. In Sultani's study the HUVECs were frozen at a concentration of 1-2 million cells per ml, and while Sultani described numerous aspects of the cryopreservation protocol that were adjusted and optimized, the cell freezing density was not altered. Similarly, a publication by Polchow et al. entitled “Cryopreservation of human vascular umbilical cord cells under good manufacturing practice conditions for future cell banks” described preparation of HUVECs for clinical use and described freezing of the HUVECs at concentrations of 1 million cells per ml in freezing media containing 10% DMSO and 20% human serum albumin (HSA). See Polchow et al., 2012, Journal of Translational Medicine, Vol. 10, 98, p. 1-17. And numerous other papers and cell culture guides describe cryopreservation of ECs and HUVECs at concentrations ranging from 0.5 to 5 million cells per ml. (See, for example, Lehle et al., “Cryopreservation of human endothelial cells for vascular tissue engineering,” 2005, Cryobiology, Vol. 50, p. 154-161; Lonza, “Clonetics™ Endothelial Cell System; Technical Information & Instructions,” 2002, www.lonza.com; Pegg., “Cryopreservation of vascular endothelial cells as isolated cells and as monolayers,” 2002, Cryobiology, Vol. 44, p. 46-53;

Reardon et al., “Investigating membrane and mitochondrial cryobiological responses of HUVEC using interrupted cooling protocols,” 2015, Cryobiology, Vol. 71, p. 306-317; and von Bomhard et al., “Cryopreservation of Endothelial Cells in Various Cryoprotective Agents and Media— Vitrification versus Slow Freezing Methods,” 2016, PLoS ONE, Vo. 11. No. 2).

One might have expected the extremely high cell freezing densities that we describe herein to be detrimental—for example as a result of limiting the per cell availability of cryopreservatives and/or nutrients or due physical stress on the cells. Indeed, prior studies with various cell types have found detrimental effects of freezing cells at high densities. For example, a study by De Loecher et al. found that increasing the concentration of red blood cells during cryopreservation increased hemolysis post-thaw, and increasing the concentration of hepatocytes during cryopreservation dramatically reduced both the viability and metabolic activity of the hepatocytes post-thaw. See De Loecher et al., “Effects of Cell Concentration on Viability and Metabolic Activity During Cryopreservation,” 1998, Cryobiology, Vol. 37(2), p. 103-109. The authors of De Loecher et al. hypothesized that this may have resulted from cell stress produced by unphysiological cell—cell contacts occurring during the freezing and thawing cycle. Surprisingly, however, we found no evidence of detrimental effects when we froze our endothelial cells at densities 20 to 100-fold higher than typical endothelial cell freezing concentrations. On the contrary, our thawed ECs exhibited expected or better than expected cell viability, cell proliferation, and ability to expand co-cultured CD34+ cord blood cells.

The extremely high cell freezing densities that we describe herein provide numerous advantages. One advantage is that sufficient ECs (e.g. HUVECs) for infusion into a patient can be provided in a single container—avoiding the need for combining the contents of multiple containers of ECs. Another major advantage is that the thawed EC-containing compositions can be diluted to directly yield a therapeutically useful EC composition—i.e. an EC composition having a suitable number and density of ECs for administration to a patient in a solution that is suitable for administration to a patient. This can be achieved because, at the level of dilution needed/used, the concentration of cryopreservatives in the diluted composition is safe for administration to a patient. This eliminates the need to remove the cryopreservatives by performing centrifugation and resuspension steps and also means that the need to transfer the ECs to different containers can be eliminated if desired. For example, if desired a suitable diluent composition can be added directly to the container in which the ECs were frozen and thawed to generate the final EC composition—which may then be transferred directly to a patient in a closed system.

Building on the discovery that ECs, such as HUVECs, can be frozen and thawed at the high densities described herein, we also sought to develop various other improvements to our compositions and methods for freezing and thawing ECs, and administering them to patients, including optimizing the level of human serum albumin (HSA) used.

The improved compositions and methods that we developed for cryopreservation and use of ECs are described further below and elsewhere throughout this patent disclosure.

Accordingly, in some aspects the present invention provides various compositions comprising endothelial cells (ECs) in freezing media. These compositions may exist at different temperatures and in different states—for example they may exist in a pre-freezing state, in a frozen/cryopreserved state, or in a thawed (post freezing/post cryopreservation) state.

For example, in one embodiment the present invention provides a composition comprising (a) endothelial cells (ECs) at a high density, and (b) a freezing medium comprising an effective amount of a cryopreservative. In some of such embodiments the ECs are at a density of from about 50 million cells per ml to about 150 million cells per ml. In some of such embodiments the ECs are at a density of from about 75 million cells per ml to about 125 million cells per ml. In some embodiments the ECs are at a density of about 100 million cells per ml.

In some embodiments the compositions may comprise any desired ECs. In some embodiments the compositions comprise ECs from a tissue selected from lung, liver, kidney, bladder, pancreas, thymus, intestine, testis, ovary, uterus, heart, nervous system, brain, spinal cord, eye, retina, skin, adipose tissue, lymphatic tissue, bone marrow, placenta and umbilical cord. In some embodiments the ECs in the compositions are umbilical vein endothelial cells (UVECs). In some embodiments the ECs in the compositions are human umbilical vein endothelial cells (HUVECs).

In some embodiments the ECs in the compositions are E4ORF1+ECs. In such embodiments the ECs comprise a recombinant nucleotide sequence that encodes an adenovirus E4ORF1 protein. In some embodiments such nucleotide sequence is operatively linked to a heterologous promoter. In some embodiments such nucleotide sequence is within a vector, such as a retroviral vector. In some embodiments such nucleotide sequence is within a lentiviral vector. In some embodiments such nucleotide sequence is within a Maloney murine leukemia virus (MMLV) vector. In some embodiments the E4ORF1 is human adenovirus type 5 E4ORF1. In some embodiments the ECs do not comprise an entire adenoviral E4 region. In some embodiments the ECs do not comprise an E4ORF2, E4ORF3, E4ORF4, E4ORF5 or E4ORF6 coding sequence or amino acid sequence.

In some embodiments the ECs in the compositions may comprise other recombinant nucleotide sequences. For example, in some embodiments the ECs in the compositions may comprise recombinant nucleotide sequences that encode and express BMP4 (i.e. they may be BMP4+ECs). Similarly, in some embodiments the ECs in the compositions may comprise recombinant nucleotide sequences that encode ETS transcription factors, such as ETV2 (i.e. they may be ETV2+ECs).

In some embodiments the compositions also comprise human serum albumin (HSA). In some embodiments the compositions comprise from about 10% to about 20% HSA. In some embodiments the compositions comprise about 10% HAS.

In some embodiments the compositions may comprise any suitable cryopreservative. In some embodiments the cryopreservative is selected from the group consisting of dimethyl sulfoxide (DMSO), ethylene glycol, propylene glycol, and glycerol. In some embodiments the cryopreservative is dimethyl sulfoxide (DMSO). For example, in some embodiments the compositions comprise from about 5% to about 10% dimethyl sulfoxide. In some embodiments the compositions comprise about 5% dimethyl sulfoxide.

In some embodiments the compositions may comprise any freezing medium that is suitable for cryopreservation of endothelial cells. Numerous such freezing media are known in the art and/or are commercially available. Some such media are described in the Examples section of this patent disclosure. In some embodiments the freezing medium is serum free.

In addition to ECs, in some embodiments the compositions of the present invention may also comprise additional cell types. In some embodiments the compositions may comprise stem cells—in addition to ECs. In some embodiments the compositions may comprise progenitor cells—in addition to ECs. In some embodiments the compositions may comprise mesenchymal stem cells—in addition to ECs. In some embodiments the compositions may comprise hematopoietic stem cells and/or hematopoietic progenitor cells—in addition to ECs. In such embodiments the hematopoietic stem cells or hematopoietic progenitor cells may be from bone marrow, peripheral blood, amniotic fluid, or umbilical cord blood. In some embodiments the compositions may comprise parenchymal cells—in addition to ECs. In some embodiments the compositions may comprise pancreatic islet cells—in addition to ECs. In some embodiments the compositions may comprise neural cells—in addition to ECs. In some embodiments the compositions may comprise glial cells—in addition to ECs.

In some embodiments, the various compositions described herein may be provided in a container suitable for use in freezing cells (i.e. a freezing container). In some embodiments the composition may be provided in a cryovial. In some embodiments the composition may be provided in a cryobag. It is particularly desirable for the compositions to be provided in a container (e.g. a cryovial or cryobag) that is adapted so that its contents (e.g. thawed ECs) can be aseptically removed from the container, diluted to form a final clinical therapeutic product, and administered to a patient in a closed system to reduce the risk or contamination.

Examples of commercially available freezing containers that can be used include, but are not limited to, Crystal Zenith® cryovials manufactured by Daikyo and Briostor™ Transfer/Freezing Bag Sets manufactured by Pall Medical.

The various compositions described above and elsewhere herein can be used in any situation in which ECs are typically used and/or in which ECs need to be frozen/cryopreserved—including for research purposes and/or for therapeutic purposes.

In some embodiments the present invention provides various methods of preparing therapeutic compositions suitable for administration to subjects (such as human subjects). In some embodiments such methods involve diluting one of the compositions described herein (e.g. that has previously been frozen and subsequently thawed) with a physiological saline solution in order to yield a therapeutic composition comprising an EC cell concentration after dilution that is suitable for administration to a subject in a therapeutic method. For example, in some such embodiments one of the compositions described herein is diluted with a physiological saline to yield a final EC concentration of from about 3 million cells per ml to about 5 million cells per ml. In some such embodiments the physiological saline used for dilution may comprise various agents that are desired components of the final therapeutic composition. Such components may include, for example, dextran (e.g. dextran40) and/or HSA. In some such embodiments such agents may not be in the physiological saline but by nonetheless be added to the composition. In some embodiments, dextran and/or HSA are added (whether in the saline or otherwise) such that, after dilution, the therapeutic composition comprises about 8% dextran (e.g. dextran40) and about 4% HSA.

In other aspects the present invention provides various methods for freezing endothelial cells.

For example, in one embodiment the present invention provides a method of freezing endothelial cells, the method comprising: (a) suspending endothelial cells (ECs) at a density of from about 50 million cells per ml to about 150 million cells per ml in a freezing medium, wherein the freezing medium comprises an effective amount of a cryopreservative, thereby creating a freezing composition, and (b) subsequently subjecting the freezing composition to a gradual decrease in temperature to at least about −80° C. to −90° C. In some such methods the temperature is decreased at a rate of about 1° C. per minute—for example using a controlled rate freezer. In some such methods the freezing composition is subsequently transferred to liquid nitrogen.

In some embodiments the freezing methods may be performed using any desired ECs. In some embodiments the freezing methods are performed using human umbilical vein endothelial cells (HUVECs).

In some embodiments the freezing methods may also involve adding human serum albumin (HSA) to the freezing composition. In some embodiments the freezing methods involve adding HSA to yield a final concentration of about 10% to about 20% HSA in the freezing compositions. In some embodiments the freezing methods involve adding HSA to yield a final concentration of about 10% HSA in the freezing compositions.

In some embodiments the freezing methods may performed using any suitable cryopreservative. In some embodiments the cryopreservative is selected from the group consisting of dimethyl sulfoxide, ethylene glycol, propylene glycol, and glycerol. In some embodiments the cryopreservative is dimethyl sulfoxide. For example, in some embodiments the freezing methods may performed using from about 5% to about 10% dimethyl sulfoxide in the freezing composition. In some embodiments the freezing methods may performed using about 10% dimethyl sulfoxide in the freezing composition.

In some embodiments the freezing methods may performed using any freezing medium that is suitable for cryopreservation of endothelial cells.

In some embodiments the freezing methods may performed using ECs that are E4ORF1+. In such embodiments the ECs will typically comprise a recombinant nucleotide sequence that encodes an adenovirus E4ORF1 protein. In some embodiments such nucleotide sequence is operatively linked to a heterologous promoter. In some embodiments such nucleotide sequence is within a vector, such as a retroviral vector. In some embodiments such nucleotide sequence is within a lentiviral vector. In some embodiments such nucleotide sequence is within a Maloney murine leukemia virus (MMLV) vector. In some embodiments the E4ORF1 is human adenovirus type 5 E4ORF1. In some embodiments the ECs do not comprise an entire adenoviral E4 region. In some embodiments the ECs do not comprise an E4ORF2, E4ORF3, E4ORF4, E4ORF5 or E4ORF6 coding sequence or amino acid sequence.

In some embodiments the freezing methods may performed using additional cell types—in addition to ECs. For example, in some embodiments the freezing methods may performed using hematopoietic stem cells and/or hematopoietic progenitor cells—in addition to ECs. In such embodiments the hematopoietic stem cells or hematopoietic progenitor cells may be from bone marrow, peripheral blood, amniotic fluid, or umbilical cord blood.

In some embodiments, the freezing methods are performed using a container suitable for use in freezing cells (i.e. a freezing container)—in which the ECs are maintained during the various method steps. In some embodiments, the freezing methods are performed using a cryovial. In some embodiments, the freezing methods are performed using a cryobag. It is particularly desirable for the freezing method to be performed using a container (e.g. a cryovial or cryobag) that is adapted so that its contents (e.g. thawed ECs) can be aseptically removed from the container, diluted to form a final clinical therapeutic product, and administered to a patient in a closed system to reduce the risk or contamination.

These and other embodiments of the invention are described further in other sections of this patent disclosure. In addition, as will be apparent to those of skill in the art, certain modifications and combinations of the various embodiments described herein fall within the scope of the present invention.

### BRIEF DESCRIPTION OF THE DRAWINGS

FIG. 1. Graph showing total cell counts of E4ORF1+HUVECs at 0, 2, 4, 6, 24, 48 and 72 hours post-thaw. Cells were frozen at a concentration of 1×10⁸ (i.e., 100 million) cells per ml in 2 ml cryovials using the rate-controlled freezing program described in Example 1. “Initial” refers to pre-freeze data.

FIG. 2. Graph showing viability of E4ORF1+HUVECs at 0, 2, 4, 6, 24, 48 and 72 hours post-thaw. Cells were frozen at a concentration of 1×10⁸ cells per ml in 2 ml cryovials using the rate-controlled freezing program described in Example 1. “Initial” refers to pre-freeze data.

FIG. 3. Graph showing viable cell counts of E4ORF1+HUVECs at 0, 2, 4, 6, 24, 48 and 72 hours post-thaw. Cells were frozen at a concentration of 1×10⁸ cells per ml in 2 ml cryovials using the rate-controlled freezing program described in Example 1. “Initial” refers to pre-freeze data.

FIG. 4. Graph showing viable cell recovery of E4ORF1+HUVECs at 0, 2, 4, 6, 24, 48 and 72 hours post-thaw. Cells were frozen at a concentration of 1×10⁸ cells per ml in 2 ml cryovials using the rate-controlled freezing program described in Example 1.

FIG. 5. Bar charts showing percentage viability (left panel) and percentage recovery (right panel) of E4ORF1+HUVECs frozen at either 1.3×10⁷ (i.e., 13 million) cells/ml or 1×10⁸ (i.e., 100 million) cells/ml, as indicated, in in a freezing medium comprising 5% DMSO and 20% human serum albumin (HSA) in 2-mL cryovials using the HUVEC freezing program described in Example 1. Cells were cryopreserved for at least 24 hours in liquid nitrogen before thawing, diluting at a 1:20 ratio in a dilution buffer containing 8.3% dextran and 4.2% HSA without any centrifugation/pelleting or rinsing to remove cryopreservative.

### DETAILED DESCRIPTION

The “Summary of the Invention” and “Claims” sections of this patent disclosure describe the main embodiments of the invention. This “Detailed Description” section provides certain additional description relating to the compositions and methods of the present invention, and is intended to be read in conjunction with all other sections of this patent disclosure. Furthermore, and as will be apparent to those in the art, the different embodiments described throughout this patent disclosure can be, and are intended to be, combined in various different ways. Such combinations of the specific embodiments described herein are intended to fall within the scope of the present invention

Definitions & Abbreviations

Certain definitions and abbreviations are provided below. Other terms or phrases may be defined elsewhere in this patent disclosure or may have meanings that are clear frm the context in which they are used. Unless defined otherwise herein, or unless some other meaning is clear from their use in context herein, all technical and scientific terms and abbreviations used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10.

Wherever embodiments are described with the language “comprising,” otherwise analogous embodiments described in terms of “consisting of” and/or “consisting essentially of” are included.

As used herein, the terms “about” and “approximately,” when used in relation to numerical values, mean within + or −10% of the stated value.

As used herein, the abbreviation “EC(s)” refers to endothelial cell(s).

As used herein, the abbreviation “E4ORF1” refers to open reading frame (ORF) 1 of the early 4 (E4) region of an adenovirus genome, or a polypeptide/protein encoded by that ORF (whether the gene or the protein is referred to will be clear from the context of use).

As used herein, the abbreviation “E4ORF1+” is used to refer to cells engineered to express E4ORF1. For example, the abbreviation “E4ORF1+HUVECs” is used to refer to human umbilical cord endothelial cells (HUVECs) engineered to express E4ORF1. E4ORF1+ cells contain a recombinant E4ORF1 nucleic acid molecule and express the E4ORF1 protein.

The term “culturing” as used herein, refers to the propagation of cells on or in media of various kinds. “Co-culturing” refers to the propagation of two or more distinct types of cells on or in media of various kinds.

As used herein the term “effective amount” refers to an amount of a specified agent (e.g., a cryopreservative) or a specified cell population (e.g. E4ORF1+HUVECs) that is sufficient to achieve one or more of the outcomes described herein. For example, an effective amount of a cryopreservative is an amount that results in effective cell freezing and effective cell recovery following freezing. An appropriate “effective amount” in any individual case may be determined empirically, for example using standard techniques known in the art. Furthermore, an “effective amount” may be determined using assays such as those described in the Examples section of this patent disclosure to assess effects on cell freezing and/or on recovery from cell freezing. For all of the embodiments described herein that involve compositions comprising, or methods of using, specified agents or specified cell populations, in some embodiments the amount of the agent(s) or cell population(s) is any effective amount. For example, if no specific amount is specified, the amount may be any effective amount.

The term “engineered” when used in relation to cells (typically endothelial cells such as HUVECs) herein refers to cells that have been engineered by man to result in the recited phenotype (e.g. E4ORF1⁺) or to express a recited nucleic acid molecule or polypeptide. The term “engineered cells” is not intended to encompass naturally occurring cells, but is, instead, intended to encompass, for example, cells that comprise a recombinant nucleic acid molecule, or cells that have otherwise been altered artificially (e.g. by genetic modification), for example so that they express a polypeptide that they would not otherwise express.

The terms “frozen” and “cryopreserved” (and grammatical variations thereof) are used interchangeably herein, and are used in accordance with their usual meaning in the art.

The terms “genetic modification” and/or “genetically modified” and/or “gene-modified” refer to any addition, deletion, alteration or disruption of or to a nucleotide sequence or to a cell's genome or to a cell's content of genetic material. In some embodiments, the endothelial cells described herein may, in addition to being genetically modified to provide a nucleic acid molecule that encodes E4ORF1, may also comprise one or more other genetic modifications—as desired. The term “genetic modification” and the above related terms encompass both transient and stable genetic modification and encompass the use of various different gene delivery vehicles and methods including, but not limited to, transduction (viral mediated transfer of nucleic acid to a recipient, either in vivo or in vitro), transfection (uptake by cells of isolated nucleic acid), liposome mediated transfer and others means of gene delivery that are well known in the art.

As used herein the term “isolated” refers to a cell population, product, compound, or composition which is separated from at least one other cell population, product, compound, or composition with which it is associated in its usual state, such as in its naturally occurring state in the body of a living subject.

As used herein, the term “recombinant” refers to nucleic acid molecules that are isolated, generated and/or designed by man (including by a machine) using methods of molecular biology and genetic engineering (such as molecular cloning), and that either comprise nucleotide sequences that do not exist in nature, or are comprised within nucleotide sequences that do not exist in nature, or are provided in association with nucleotide sequences that they would not be associated with in nature, or that are provided in the absence of nucleotide sequences with which they would ordinarily be associated in nature. Thus, recombinant nucleic acid molecules are to be distinguished from nucleic acid molecules that exist in nature—for example in the genome of an organism. For example, a nucleic acid molecule that comprises a complementary DNA or “cDNA” copy of an mRNA sequence, without any intervening intronic sequences such as would be found in the corresponding genomic DNA sequence, would thus be considered a recombinant nucleic acid molecule. By way of a further example, a recombinant E4ORF1 nucleic acid molecule might comprise an E4ORF1 coding sequence operatively linked to a promoter and/or other genetic elements with which that coding sequence is not ordinarily associated in a naturally-occurring adenovirus genome, or in the absence of absence of nucleotide sequences with which it would ordinarily be associated in an adenovirus genome.

The term “subject” includes mammals—such as humans and non-human primates, as well as other mammalian species including rabbits, rats, mice, cats, dogs, horses, cows, sheep, goats, pigs and the like. In some embodiments the subjects are mammalian subjects. In some embodiments the subjects are humans. In some embodiments the subjects are non-human primates.

The terms “patient” and “human subject” may be used interchangeably herein.

E4ORF 1

Several of the embodiments of the present invention described herein involve engineered endothelial cells (ECs) that are E4ORF1+. The “E4ORF1” polypeptide is encoded by open reading frame (ORF) 1 of the early 4 (E4) region of the adenovirus genome. E4ORF1+ECs for use in accordance with the present invention typically comprise a recombinant nucleic acid molecule that contains an E4ORF1 coding sequence operatively linked to a promoter suitable for expression of the E4ORF1 coding sequence in endothelial cells.

E4ORF1 amino acid sequences and nucleotide sequences are known in the art. Any such sequences may be used in accordance with the present invention. In some embodiments the E4ORF1 polypeptide may be from any suitable adenovirus type or strain, such as human adenovirus type 2, 3, 5, 7, 9, 11, 12, 14, 34, 35, 46, 50, or 52. In some embodiments the polypeptide sequence used is from human adenovirus type 5. Amino acid sequences of such adenovirus polypeptides, and nucleic acid sequences that encode such polypeptides, are well known in the art and available in well-known publicly available databases, such as the Genbank database. For example, suitable sequences include the following: human adenovirus 9 (Genbank Accession No. CA105991), human adenovirus 7 (Genbank Accession No. AAR89977), human adenovirus 46 (Genbank Accession No. AAX70946), human adenovirus 52 (Genbank Accession No. ABK35065), human adenovirus 34 (Genbank Accession No. AAW33508), human adenovirus 14 (Genbank Accession No. AAW33146), human adenovirus 50 (Genbank Accession No. AAW33554), human adenovirus 2 (Genbank Accession No. AP.sub.--000196), human adenovirus 12 (Genbank Accession No. AP.sub.--000141), human adenovirus 35 (Genbank Accession No. AP.sub.--000607), human adenovirus 7 (Genbank Accession No. AP.sub.--000570), human adenovirus 1 (Genbank Accession No. AP.sub.--000533), human adenovirus 11 (Genbank Accession No. AP.sub.--000474), human adenovirus 3 (Genbank Accession No. ABB 17792), and human adenovirus type 5 (Genbank accession number D12587). In one embodiment the E4ORF1 sequence used is that having NCBI accession number AZR66741.1. In one embodiment the E4ORF1 sequence used is that having NCBI accession number AP 000232.1. In one embodiment the E4ORF1 sequence used is has the amino acid sequence

(SEQ ID NO. 1)
MAAAVEALFVVLEREGAILPRQEGFSGVYVFFSPINFVIPPMGAVMLSL
RLRVCIPPGYFGRFLALTDVNQPDVFTESYIMTPDMTEELSVVLFNHGD
QFFYGHAGMAVVRLMLIRVVFPVVRQASNV.

In some embodiments the E4ORF1 polypeptide may have an amino acid sequence, or may be encoded by a nucleic acid sequence, that is a variant, derivative, mutant, or fragment of any of the specific sequences provided herein or known in the art provided that such variants, derivatives, mutants, or fragments are, or encode, a polypeptide that has one or more of the functional properties of adenovirus E4ORF1 known in the art (for example as described in U.S. Pat. No. 8,465,732) or described herein. In some embodiments, the variants, derivatives, mutants, or fragments have about an 85% identity to the known sequence, or about an 88%, 89%, 90%, 91%, 92%, 93%, 94%, 95%, 96%, 97%, 98%, or 99% sequence identity to the known sequence. In some embodiments, a variant, derivative, mutant, or fragment of a known nucleotide sequence is used that varies in length by about 50 nucleotides, or about 45 nucleotides, or about 40 nucleotides, or about 35 nucleotides, or about 30 nucleotides, or about 28 nucleotides, 26 nucleotides, 24 nucleotides, 22 nucleotides, nucleotides, 18 nucleotides, 16 nucleotides, 14 nucleotides, 12 nucleotides, 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide relative to the known nucleotide sequence. In some embodiments, a variant, derivative, mutant, or fragment of a known amino sequence is used that varies in length about 50 amino acids, or about 45 amino acids, or about 40 amino acids, or about 35 amino acids, or about 30 amino acids, or about 28 amino acids, 26 amino acids, 24 amino acids, 22 amino acids, 20 amino acids, 18 amino acids, 16 amino acids, 14 amino acids, 12 amino acids, 10 amino acids, 9 amino acids, 8 amino acids, 7 amino acids, 6 amino acids, 5 amino acids, 4 amino acids, 3 amino acids, 2 amino acids, or 1 amino acid relative to the known amino acid sequence.

In some embodiments E4ORF1 sequences are used without other sequences from the adenovirus E4 region—for example not in the context of the entire E4 region or not together with other ORFs in the E4 region. However, in other embodiments E4ORF1 may be used in conjunction with one or more other ORFs from the E4 region, such as E4ORF2, E4ORF3, E4ORF4, E4ORF5 or E4ORF6/7 sequences. For example, although E4ORF1 sequences can be used in constructs (such as a viral vectors) that contain other sequences, genes, or coding regions (such as promoters, marker genes, antibiotic resistance genes, and the like), in certain embodiments, the E4ORF1 sequences are used in constructs that do not contain the entire E4 region, or that do not contain other ORFs from the entire E4 region, such as E4ORF2, E4ORF3, E4ORF4, and/or E4ORF5.

E4ORF1 encoding sequences can be present in constructs or vectors that contain various other sequences, genes, or coding regions, for example, promoters, enhancers, antibiotic resistance genes, reporter genes or expression tags (such as, for example nucleotides sequences encoding GFP), or any other nucleotide sequences or genes that might be desirable.

E4ORF1-encoding nucleic acid molecules can be under the control of one or more promoters to allow for expression. Any promoter able to drive expression of the E4ORF1 nucleic acid sequences in endothelial cells can be used. Examples of suitable promoters include, but are not limited to, the CMV, SV40, RSV, HIV-Ltr, and MML promoters. The promoter can also be a promoter from the adenovirus genome, or a variant thereof. For example, in some embodiments the promoter may be a promoter that drives expression of E4ORF1 in nature in an adenovirus genome. However, in other embodiments the promoter is not one that drives expression of E4ORF1 in nature in an adenovirus genome.

The E4ORF1-encoding sequences may comprise naturally occurring nucleotides, synthetic nucleotides, or a combination thereof. For example, in some embodiments the nucleic acid molecules of the invention can comprise RNA, such as synthetic modified RNA that is stable within cells and can be used to direct protein expression/production directly within cells. In other embodiments the E4ORF1-encoding sequences can comprise DNA. In embodiments where DNA is used, the DNA sequences may be operably linked to one or more suitable promoters and/or regulatory elements to allow (and/or facilitate, enhance, or regulate) expression within cells, and may be present in one or more suitable vectors or constructs.

The E4ORF1-encoding sequences can be introduced into endothelial cells using any suitable system known in the art, including, but not limited to, transfection techniques and viral-mediated transduction techniques. Transfection methods that can be used in accordance with the present invention include, but are not limited to, liposome-mediated transfection, polybrene-mediated transfection, DEAE dextran-mediated transfection, electroporation, calcium phosphate precipitation, microinjection, and micro-particle bombardment. Viral-mediated transduction methods that can be used include, but are not limited to, lentivirus-mediated transduction, adenovirus-mediated transduction, retrovirus-mediated transduction, adeno-associated virus-mediated transduction and herpesvirus-mediated transduction.

In some embodiments the E4ORF1-encoding sequences are in a vector. In some embodiments the E4ORF1-encoding sequences are in a viral vector. In some embodiments the E4ORF1-encoding sequences are in a lentiviral vector. In some embodiments the E4ORF1-encoding sequences are in an adenoviral vector. In some embodiments the E4ORF1-encoding sequences are in adeno-associated virus vector. In some embodiments the E4ORF1-encoding sequences are in a retroviral vector. In some embodiments the E4ORF1-encoding sequences are in a Moloney murine leukemia virus (MMLV) vector (a type of retroviral vector).

In some embodiments the compositions of the present invention comprise both E4ORF1+ and E4ORF1-negative endothelial cells. In some embodiments at least about 75% of the endothelial cells in the composition are E4ORF1+. In some embodiments at least about 80% of the endothelial cells in the composition are E4ORF1+. In some embodiments at least about 85% of the endothelial cells in the composition are E4ORF1+. In some embodiments at least about 90% of the endothelial cells in the composition are E4ORF1+. In some embodiments at least about 95% of the endothelial cells in the composition are E4ORF1+. In some embodiments at least about 98% of the endothelial cells in the composition are E4ORF1+. In some embodiments at least about 99% of the endothelial cells in the composition are E4ORF1+.

In some embodiments the compositions of the present invention comprise endothelial cells that, on average across all of the endothelial cells in the composition, comprise less than one copy of a genomically integrated E4ORF1 coding sequence per cell. In some embodiments the compositions of the present invention comprise endothelial cells that, on average across all of the endothelial cells in the composition, comprise about one copy of a genomically integrated E4ORF1 coding sequence per cell. In some embodiments the compositions of the present invention comprise endothelial cells that, on average across all of the endothelial cells in the composition, comprise more than one copy of a genomically integrated E4ORF1 coding sequence per cell. In some embodiments the compositions of the present invention comprise endothelial cells that, on average across all of the endothelial cells in the composition, comprise about 0.7 copies of a genomically integrated E4ORF1 coding sequence per cell. In some embodiments the compositions of the present invention comprise endothelial cells that, on average across all of the endothelial cells in the composition, comprise about 0.8 copies of a genomically integrated E4ORF1 coding sequence per cell. In some embodiments the compositions of the present invention comprise endothelial cells that, on average across all of the endothelial cells in the composition, comprise about 0.9 copies of a genomically integrated E4ORF1 coding sequence per cell. In some embodiments the compositions of the present invention comprise endothelial cells that, on average across all of the endothelial cells in the composition, comprise about 1.0 copies of a genomically integrated E4ORF1 coding sequence per cell. In some embodiments the compositions of the present invention comprise endothelial cells that, on average across all of the endothelial cells in the composition, comprise about 1.1 copies of a genomically integrated E4ORF1 coding sequence per cell. In some embodiments the compositions of the present invention comprise endothelial cells that, on average across all of the endothelial cells in the composition, comprise about 1.2 copies of a genomically integrated E4ORF1 coding sequence per cell. In some embodiments the compositions of the present invention comprise endothelial cells that, on average across all of the endothelial cells in the composition, comprise about 1.3 copies of a genomically integrated E4ORF1 coding sequence per cell. In some embodiments the compositions of the present invention comprise endothelial cells that, on average across all of the endothelial cells in the composition, comprise about 1.4 copies of a genomically integrated E4ORF1 coding sequence per cell. In some embodiments the compositions of the present invention comprise endothelial cells that, on average across all of the endothelial cells in the composition, comprise about 1.5 copies of a genomically integrated E4ORF1 coding sequence per cell.

In some embodiments the presence of E4ORF1 coding sequences can be confirmed and/or quantified using standard nucleic acid detection and/or quantification assays known in the art, such as PCR-based techniques (e.g., quantitative PCR) and sequencing-based techniques (e.g., quantitative next generation sequencing-based techniques). In some embodiments the presence of E4ORF1 polypeptides can be confirmed and/or quantified using standard protein detection and/or quantification assays known in the art, such as antibody-based techniques. In some embodiments the expression of functional E4ORF1 polypeptide (or an appropriate amount of functional E4ORF1 polypeptide can be confirmed and/or quantified using functional assays (e.g., in vitro or in vivo assays) for any of the functional properties of E4ORF1-expressing endothelial cells that are known in the art (such as any of those described in U.S. Pat. No. 8,465,732). In some of such embodiments the results of any of such assays can be compared between batches of E4ORF1+endothelial cells (e.g., between a test batch and a control batch having known E4ORF1 properties), for example to assess consistency and/or to make any adjustments based thereon.

The handling, manipulation, and expression of E4ORF1 sequences in endothelial cells may be performed using conventional techniques of molecular biology and cell biology. Such techniques are well known in the art. For example, one may refer to the teachings of Sambrook, Fritsch and Maniatis eds., “Molecular Cloning A Laboratory Manual, 2nd Ed.,

Cold Springs Harbor Laboratory Press, 1989); the series Methods of Enzymology (Academic Press, Inc.), or any other standard texts for guidance on suitable techniques to use in handling, manipulating, and expressing nucleotide and/or amino acid sequences. Additional aspects relevant to the handling and expression of E4ORF1 sequences in endothelial cells are described in U.S. Pat. No. 8,465,732, the contents of which are hereby incorporated by reference.

Endothelial Cells

In some embodiments the endothelial cells (ECs) described herein can be derived from any suitable source of vascular endothelial cells known in the art. In some embodiments the endothelial cells are primary endothelial cells. In some embodiments the endothelial cells are mammalian cells, such as human or non-human primate cells, or rabbit, rat, mouse, goat, pig, or other mammalian cells. In some embodiments the endothelial cells are primary human endothelial cells. In some embodiments the endothelial cells are umbilical vein endothelial cells (UVECs), such as human umbilical vein endothelial cells (HUVECs). In some embodiments the endothelial cells are adipose ECs. In some embodiments the endothelial cells are skin ECs. In some embodiments the endothelial cells are cardiac ECs. In some embodiments the endothelial cells are kidney ECs. In some embodiments the endothelial cells are lung ECs. In some embodiments the endothelial cells are liver ECs. In some embodiments the endothelial cells are bone marrow ECs. Other suitable endothelial cells that can be used include those described previously as being suitable for E4ORF1-expression in U.S. Pat. No. 8,465,732, the contents of which are hereby incorporated by reference.

In some embodiments the endothelial cells are gene-modified such that they comprise one or more genetic modifications. For example, in some embodiments the endothelial cells are engineered to express E4ORF1. In some embodiments the endothelial cells may also be engineered to express ETV2. Indeed, in each of the embodiments described throughout this patent disclosure where the ECs express E4ORF1, the ECs may also express ETV2. Similarly, in some embodiments the endothelial cells may also be engineered to express BMP4. Indeed, in each of the embodiments described throughout this patent disclosure where the ECs express E4OR1, the ECs may also express BMP4.

Furthermore, in some embodiments the ECs described herein may comprise a corrected version of a gene known to be involved in, or suspected of being involved in, a disease or disorder that affects endothelial cells, or any other gene, such as a therapeutically useful gene, that it may be desired to provide in endothelial cells or administer or deliver using engineered endothelial cells.

Methods of Use

In some embodiments, the compositions described herein can be used in various therapeutic methods, or can be used in the preparation of therapeutic compositions which can in turn be used in various therapeutic methods. Such therapeutic methods may comprise any methods for which the administration of ECs (such as HUVECs) to a subject may be desired or beneficial. In carrying out such therapeutic methods the therapeutic compositions described herein can be administered to subjects using any suitable means known in the art, for example by injection (e.g. intravenous injection, intramuscular injection, subcutaneous injection, local injection), by infusion (e.g. by intravenous infusion, subcutaneous infusion, local infusion), or by surgical implantation. The therapeutic compositions can be administered in a single dose or in multiple doses. The skilled artisan will be able to select a suitable route of administration and a suitable schedule of administration depending on the particular situation.

In some embodiments the therapeutic compositions described herein may comprise, or be administered together with, compositions comprising one or more additional cell types. Such additional cell types may be, for example stem or progenitor cells, such as hematopoietic stem cells, hematopoietic progenitor cells, c-kit+Scal+ hematopoietic stem cells, lymphoid progenitor cells, CD4-CD8-CD44+CD25-ckit+ cells, early thymic progenitors, CD4-CD8-CD44+CD25-ckit- cells or DN1 cells.

Cell Culture & Cryopreservation Methods

Methods of culturing cells are well known in the art and any suitable cell culture methods can be used. For example, ECs can be cultured using methods known to be useful for culturing other endothelial cells, or, methods known to be useful for culturing E4ORF1-expressing endothelial cells, for example as described in U.S. Pat. No. 8,465,732, the contents of which are hereby incorporated by reference. In some embodiments the ECs can be cultured in the absence of serum, or in the absence of exogenous growth factors, or in the absence of both serum and exogenous growth factors.

Exemplary cryopreservation protocols are described herein—including in the Examples section of this patent disclosure. In addition, several methods for cryopreservation of ECs (including HUVECs) are known in the art and can be used in connection with the present invention. In particular, reference is made to the EC/HUVEC cryopreservation methods described in Lehle et al., “Cryopreservation of human endothelial cells for vascular tissue engineering.” Cryobiology 50 (2005) 154-161; Lehle et al., “Identification and Reduction of Cryoinjury in Endothelial Cells: A First Step toward Establishing a Cell Bank for Vascular Tissue Engineering.” Tissue Engineering Volume 12, Number 12, 2006; Lonza; “Clonetics™ Endothelial Cell System—Technical Information & Instructions,” www.lonza.com; 2018, Technical Information & Instructions; Marquez-Curtis et al., “Beyond membrane integrity: Assessing the functionality of human umbilical vein endothelial cells after cryopreservation.” Cryobiology 72 (2016) 183-190; Pegg., “Cryopreservation of vascular endothelial cells as isolated cells and as monolayers.” Cryobiology 44 (2002) 46-53; Polchow et al., “Cryopreservation of human vascular umbilical cord cells under good manufacturing practice conditions for future cell banks.” Journal of Translational Medicine 2012 10:98; Puzanov et al., “New Approach to Cryopreservation of Primary Noncultivated Human Umbilical Vein Endothelium in Biobanking.” Biopreservation And Biobanking; Volume 16, Number 2, 2018; Sultani, A. B. et al. “Improved Cryopreservation of Human Umbilical Vein Endothelial Cells: A Systematic Approach.” Sci. Rep. 6, 34393; (2016); Reardon et al. “Investigating membrane and mitochondrial cryobiological responses of HUVEC using interrupted cooling protocols.” Cryobiology 71 (2015) 306-317; and von Bomhard A. et al., (2016) Cryopreservation of Endothelial Cells in Various Cryoprotective Agents and Media—Vitrification versus Slow Freezing Methods. PLoS ONE 11(2)— the contents of each of which are hereby incorporated by reference.

Kits

The present invention also contemplates kits comprising the compositions described herein, or for preparing the compositions described herein, and/or for carrying out any of the methods described herein. Such kits may contain any of the components described herein, including, but not limited to, nucleotide sequences (for example those encoding E4ORF1), ECs (such as HUVECs), populations of E4ORF1+ECs (such as E4ORF1+HUVECs), means or compositions for detection of ECs (such as HUVECs) or the proteins or nucleic acid molecules expressed therein, (e.g. nucleic acid probes, antibodies, etc.), freezing media, cryopreservatives, HSA, dextran (e.g. dextran40), cryovials, cryobags, or any combination thereof. All such kits may optionally comprise instructions for use. A label may accompany the kit and may include any writing or recorded material (which may be electronic or in computer readable form) providing instructions or other information for use of the kit contents. For example, in some embodiments such kits may comprise a composition comprising E4ORF1+HUVECs in freezing media in a cryovial or cryobag and instructions for the thawing, dilution, and/or clinical use thereof.

Certain aspects of the present invention may be further described with reference to the following non-limiting Examples.

### EXAMPLES

### Example 1

### High Density Freezing of E4ORF1+HUVECs

Experiments were performed to assess the recovery and viability of E4ORF1+HUVECs that had been cryopreserved using a controlled-rate freezing program, and using various different freezing containers—including some containers that were adapted for direct dilution and aseptic delivery of cells to patients in a closed system.

E4ORF1+HUVECs were pelleted and then suspended at a concentration of either about 13 million (1.3×10 7) cells per ml or 100 million (1.0×10⁸) cells per ml in a freezing medium comprising 5% DMSO and 20% human serum albumin (HSA).

In some experiments about 0.5 mls (0.57 mls) of this cell suspension was transferred to each of several 2 ml cryovials. In some experiments about 1 ml of this cell suspension was transferred to each of several 2 ml or 5 ml cryovials or to cryobags. In some of these experiments “Crystal Zenith” cryovials manufactured by Daikyo or Briostor™ or Transfer/Freezing Bag Sets manufactured by Pall Medical were used—each of which is adapted for aseptic delivery of thawed cell products to patients in a closed system.

A rate-controlled freezing program was utilized to freeze the E4ORF1+HUVECs in freezing medium—the details of which are provided in Table 1 below.

TABLE 1
Controlled Rate Freezing Program

| Program | Protocol |
|---|---|
| HUVEC | 1. Rate Controlled Freezer powered on and cools |
| Freezing | chamber to 4° C. at a rate of 1° C. per minute. |
| Program | 2. Cells placed in the chamber, along with a probe |
| | (which is placed in a blank cryovial). |
| | 3. Rate Controlled Freezer cools at a rate of 1° C. per |
| | minute until the probe is at a temperature of 4° C. |
| | 4. Rate Controlled Freezer cools at a rate of 1° C. per |
| | minute until probe is at a temperature of −4° C. |
| | 5. Rate Controlled Freezer cools at a rate of 25° C. per |
| | minute until chamber is at a temperature of −40° C. |
| | 6. Rate Controlled Freezer ‘warms’ at a rate of 10° C. |
| | per minute until chamber is at a temperature of −12° C. |
| | 7. Rate Controlled Freezer cools at a rate of 1° C. per |
| | minute until probe is at a temperature of −40° C. |
| | 8. Rate Controlled Freezer cools at a rate of 10° C. per |
| | minute until probe is at a temperature of −90° C. |
| | 9. Cells are removed from Rate Controlled Freezer |
| | chamber and immediately transferred to LN2 storage. |

After executing the rate-controlled freezing program, frozen cells were stored in liquid nitrogen (LN2) for at least 24 hours (1 days) to 96 hours (4 days).

The E4ORF1+HUVECs were then thawed and diluted in a dextran- and HSA-containing dilution buffer (comprising dextran 40 8.3% HSA 4.2%) to yield a diluted cell concentration of approximately 3.4 million cells per ml.

Recovery of viable cells was assessed either immediately post-thaw (i.e. at 0 hours post-thaw) or after the cells had been maintained at room temperature for 2, 4, 6, 24, 48 or 72 hours post-thaw using standard methods involving staining cells with trypan blue and counting cells using a hemocytometer.

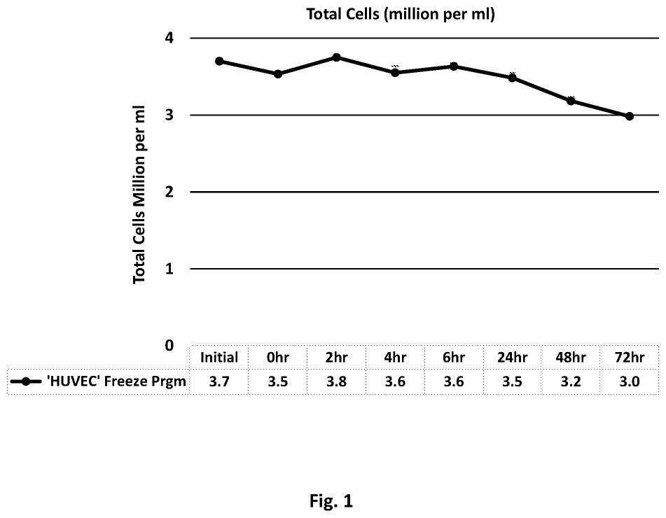

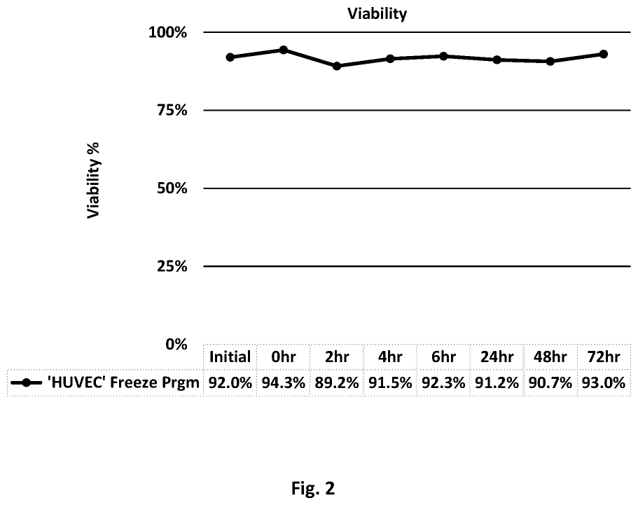

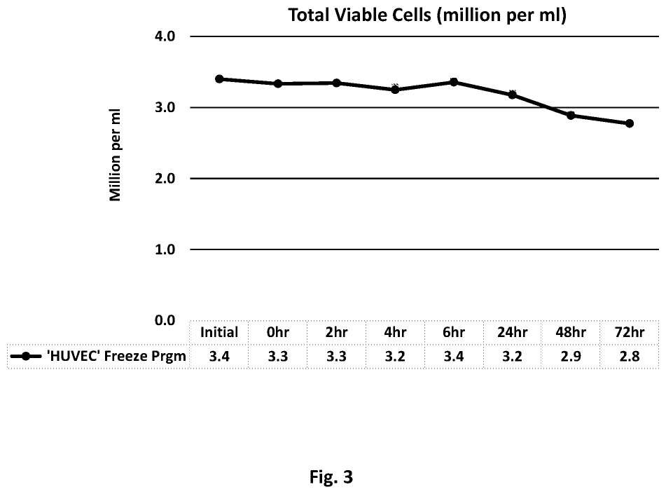

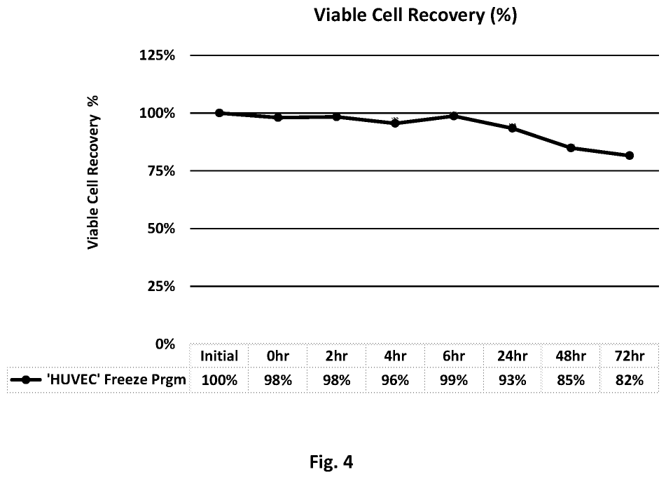

FIGS. 1-4 present, in graphical form, the total cell counts (FIG. 1), viability (FIG. 2), absolute viable cell counts (FIG. 3) and percentage viable cell recovery (FIG. 4) of E4ORF1+HUVECs at 0, 2, 4, 6, 24, 48 and 72 hours post-thaw when the cells were frozen using the rate-controlled freezing program in 2 ml cryovials (“initial” in the Figures refers to pre-freeze viability). The post-thaw viability was stable, (see FIG. 2), unexpectedly showing virtually no drop in viability over the course of the experiment.

The percentage viability and percentage recovery of E4ORF1+HUVECs frozen at either 1.3×10⁷ cells/ml or 1×10⁸ cells/ml in in a freezing medium comprising 5% DMSO and 20% human serum albumin (HSA) in either standard screw-cap cryovials, CZ cryovials, or cryobags (Briostor® Transfer/Freezing Bag Set) at either both 2-mL or 5-mL sizes HUVEC freezing program, was determined. Cells were cryopreserved for at least 24 hours in liquid nitrogen before thawing, diluting at a 1:20 ratio in a dilution buffer containing 8.3% dextran and 4.2% HSA without any centrifugation or rinsing to remove cryopreservative (as described above), subsequently assessing cell number/viability (as described above).

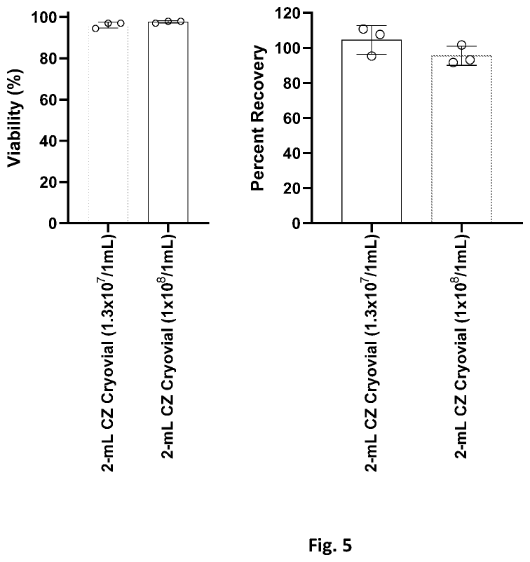

The results are shown in FIG. 5A-B and Table 2.

TABLE 2

| | Screw Cap | CZ Vial | Screw Cap | CZ vial |
|---|---|---|---|---|
| | 1.3 × 10⁷/ml | 1.3 × 10⁷/ml | 1 × 10⁸/ml | 1 × 10⁸/ml |
| Viability | 98 | 96 | 97 | 98 |
| Recovery | 102 | 105 | 78 | 96 |

The data from these studies showed:

(a) that surprisingly high levels of viability and viable cell recovery could be achieved (with either freezing protocol) even when E4ORF1+HUVECs were frozen at ultra-high cell density, with no observed decrease in viability/recovery when increasing cell densities from about 10 million cells per ml to about 100 million cells per ml,

(b) that E4ORF1+HUVECs frozen at ultra-high density can be diluted directly, without the need for removal of cryopreservatives, to generate a useable cell therapy product containing an appropriate amount and concentration of E4ORF1+HUVECs in a buffer suitable for administration to a human subject—all without any significant loss of viability, and (c) that the HUVEC freezing program we describe can be used to maintain post-thaw viability of E4ORF1+HUVECs.

### Example 2

### E4ORF1+HUVECs Frozen at High Density Can be Thawed, Diluted and Safely Administered to Human Subjects

A Phase I clinical trial was performed to assess the safety of administration of E4ORF1+HUVECs to human subjects. Subjects with chemosensitive lymphomas eligible for high dose therapy-autologous hematopoietic cell transplantation (HDT-AHCT) were enrolled.

The E4ORF1+HUVECs used in the clinical trial were supplied to clinical trials sites frozen (using methods as described herein) at a concentration of 100 million cells per ml (1×10 8 cells per ml) in a serum-free, CGMP manufactured, freezing medium (CryoStor® CS5) supplemented with Human Serum Albumin (HSA) and DMSO, such that the final concentration of HSA was 10% and the final concentration of DMSO was 5%.

At the clinical trial sites, the cells were thawed and diluted in a dilution medium (using methods as described herein)—without removal of cryopreservative—to yield a therapeutic composition comprising E4ORF1+HUVEC cells (about 5×10⁶ cells per ml), Dextran40 (˜8.3%), HSA (˜4.3%) and DMSO (˜0.25%) in saline.

The therapeutic composition was then administered intravenously to human subjects, after AHCT, in dose-escalated cohorts receiving either 5×10⁶, 10×10⁶ or 20×10⁶ cells/kg, either as a single or divided dose (in the case of divided dosing, cells were administered on day 0 and then again two days later). Supportive care therapies were administered as per site institutional guidelines.

The primary objective of the clinical trial was to assess the safety of the administered therapeutic compositions. Secondary objectives included assessment of grade ≥3 adverse events—using the NCI-CTCAEv5.0 grading system. See, Common Terminology Criteria for Adverse Events (CTCAE) Version 5.0, Published: Nov. 27, 2017, U.S. Department of Health and Human Services, National Institutes of Health, National Cancer Institute, and Freites-Martinez et al., Using the Common Terminology Criteria for Adverse Events (CTCAE—Version 5.0) to Evaluate the Severity of Adverse Events of Anticancer Therapies. Actas Dermosifiliogr (Engl Ed). 2021 January; 112(1):90-92. Under this system, Grade 1 indicates that the adverse event (AE) is mild, Grade 2 is moderate, Grade 3 is severe, Grade 4 is life-threatening, and Grade 5 is fatal (death related to the AE). Oral/gastrointestinal adverse events of grade ≥3 that were assessed included oral mucositis, nausea, vomiting or diarrhea.

Twenty-nine human subjects with systemic lymphoma were treated with a median follow up of 271 days (range 179, 566). Adverse events were generally mild/moderate and of the type and magnitude expected with HDT-AHCT. No maximum tolerated dose was established through dosing up to 20×10 6 cells/kg because the treatment was well tolerated.

The results of this Phase I study indicated that these therapeutic compositions—prepared from high density frozen E4ORF1+HUVECs using the methods and compositions described herein—could be safely administered to human subjects.

### REFERENCE LIST

ATTC; Animal Cell Culture Guide; 2014 www.atcc.org

Common Terminology Criteria for Adverse Events (CTCAE) Version 5.0, Published: Nov. 27, 2017, U.S. Department of Health and Human Services, National Institutes of Health, National Cancer Institute.

De Loecher et al., “Effects of Cell Concentration on Viability and Metabolic Activity During Cryopreservation,” 1998, Cryobiology, Vol. 37(2), p. 103-109.

Freites-Martinez et al., Using the Common Terminology Criteria for Adverse Events (CTCAE—Version 5.0) to Evaluate the Severity of Adverse Events of Anticancer Therapies. Actas Dermosifiliogr (Engl Ed). 2021 January; 112(1):90-92.

Lehle et al., “Cryopreservation of human endothelial cells for vascular tissue engineering.” Cryobiology 50 (2005) 154-161

Lehle et al., “Identification and Reduction of Cryoinjury in Endothelial Cells: A First Step toward Establishing a Cell Bank for Vascular Tissue Engineering.” TISSUE ENGINEERING Volume 12, Number 12, 2006

Lonza; Clonetics™ Endothelial Cell System; Technical Information & Instructions; www.lonza.com; 2018

Technical Information & Instructions

Marquez-Curtis et al., Beyond membrane integrity: Assessing the functionality of human umbilical vein endothelial cells after cryopreservation. Cryobiology 72 (2016) 183e190

Pegg., “Cryopreservation of vascular endothelial cells as isolated cells and as monolayers.” Cryobiology 44 (2002) 46-53

Polchow et al.: “Cryopreservation of human vascular umbilical cord cells under good manufacturing practice conditions for future cell banks.” Journal of Translational Medicine 2012 10:98.

Puzanov et al.: “New Approach to Cryopreservation of Primary Noncultivated Human Umbilical Vein Endothelium in Biobanking.” BIOPRESERVATION AND BIOBANKING; Volume 16, Number 2, 2018

Sultani, A. B. et al. “Improved Cryopreservation of Human Umbilical Vein Endothelial Cells: A Systematic Approach.” Sci. Rep. 6, 34393; (2016).

Reardon et al. “Investigating membrane and mitochondrial cryobiological responses ofHUVEC using interrupted cooling protocols.” Cryobiology 71 (2015) 306-317

U.S. Pat. No. 8,465,732.

von Bomhard A. et al., (2016) Cryopreservation of Endothelial Cells in Various Cryoprotective Agents and Media—Vitrification versus Slow Freezing Methods. PLoS ONE 11(2).

### ***

The present invention is further described by the following claims.

We claim:

1. A composition comprising E4ORF1+endothelial cells (ECs) at a density of from about 50 million cells per ml to about 150 million cells per ml in a freezing medium comprising an effective amount of a cryopreservative.

2. The composition of claim 1, wherein the ECs are human umbilical vein endothelial cells (HUVEC s).

3. The composition of claim 1 or claim 2, wherein the endothelial cells (ECs) are at a density of from about 75 million cells per ml to about 125 million cells per ml.

4. The composition of claim 1 or claim 2, wherein the endothelial cells (ECs) are at a density of about 100 million cells per ml.

5. The composition of any of the preceding claims further comprising human serum albumin (HSA).

6. The composition of any of the preceding claims further comprising about 10% human serum albumin (HSA).

7. The composition of any of the preceding claims wherein the cryopreservative is selected from the group consisting of dimethyl sulfoxide, ethylene glycol, propylene glycol, and glycerol.

8. The composition of any of the preceding claims wherein the cryopreservative is dimethyl sulfoxide.

9. The composition of any of the preceding claims comprising from about 5% to about 10% dimethyl sulfoxide.

10. The composition of any of the preceding claims comprising about 10% dimethyl sulfoxide.

11. The composition of any of the preceding claims wherein the freezing medium comprises an endothelial growth medium.

12. The composition of any of the preceding claims, wherein the ECs comprise a recombinant nucleotide sequence that encodes an adenovirus E4ORF1 protein operatively linked to a heterologous promoter.

13. The composition of claim 12, wherein the nucleotide sequence is within a vector.

14. The composition of claim 13, wherein the vector is a retroviral vector.

15. The composition of claim 14, wherein the retroviral vector is a lentiviral vector.

16. The composition of claim 15, wherein the retroviral vector is a Maloney murine leukemia virus (MMLV) vector.

17. The composition of any of the preceding claims, wherein the E4ORF1 is human adenovirus type 5 E4ORF1.

18. The composition of any of the preceding claims, wherein the ECs do not comprise an entire adenovirus E4 region.

19. The composition of any of the preceding claims, wherein the ECs do not comprise do not comprise an E4ORF2, E4ORF3, E4ORF4, E4ORF5 or E4ORF6 coding sequence or amino acid sequence.

20. A composition comprising (a) E4ORF1+ECs at a density of about 100 million cells per ml, (b) endothelial growth media, (c) about 5 to about 10% DMSO, and (d) about 10% HSA.

21. A composition comprising (a) E4ORF1+HUVECs at a density of about 100 million cells per ml, (b) endothelial growth media, (c) about 5 to about 10% DMSO, and (d) about 10% HSA.

22. A composition according to any of the preceding claims, further comprising hematopoietic stem cells or hematopoietic progenitor cells.

23. The composition of claim 22, wherein the hematopoietic stem cells or hematopoietic progenitor cells are from bone marrow.

24. The composition of claim 22, wherein the hematopoietic stem cells or hematopoietic progenitor cells are from peripheral blood.

25. The composition of claim 22, wherein the hematopoietic stem cells or hematopoietic progenitor cells are from amniotic fluid.

26. The composition of claim 22, wherein the hematopoietic stem cells or hematopoietic progenitor cells are from umbilical cord blood.

27. The composition of any of the preceding claims wherein the composition is in a freezing container.

28. The composition of claim 27, wherein the freezing container is a cryovial.

29. The composition of claim 27, wherein the freezing container is a cryobag.

30. The composition of claim 27, wherein the freezing container is adapted for aseptic transfer of its contents to a patient in a closed system.

31. A composition according to any of the preceding claims for use in the preparation of a therapeutic composition for administration to a human subject.

32. Use of a composition according to any of the preceding claims in the preparation of a therapeutic composition for administration to a human subject.

33. A method of preparing a therapeutic composition for administration to a human subject, the method comprising diluting a composition according to any of the preceding claims with a physiological saline solution, wherein the EC cell concentration after dilution is from about 3 million cells per ml to about 5 million cells per ml, thereby preparing a therapeutic composition for administration to a human subject.

34. The method of claim 33, wherein the physiological saline comprises dextran 40 and HSA at amounts such that, after dilution, the therapeutic composition comprises about 8% dextran and about 4% HSA.

35. The method of claim 33, wherein the method does not comprise any centrifugation steps.

36. The method of claim 33 or 34, wherein the method does not comprise removing the freezing medium or cyropreservative.

37. A method of freezing endothelial cells, the method comprising:
a. suspending endothelial cells (ECs) at a density of from about 50 million cells per ml to about 150 million cells per ml in a freezing medium, wherein the freezing medium comprises an effective amount of a cryopreservative, thereby creating a freezing composition, and
b. subjecting the freezing composition to a gradual decrease in temperature to about −80° C. to −90° C.

38. The method of claim 37, wherein in step (b) the temperature is decreased at a rate of about 1° C. per minute.

39. The method of claim 37 or claim 38, comprising subsequently transferring the freezing composition to liquid nitrogen.

40. The method of any of claims 37-39, wherein the endothelial cells (ECs) are at a density of from about 75 million cells per ml to about 125 million cells per ml.

41. The method of any of claims 37-39, wherein the endothelial cells (ECs) are at a density of about 100 million cells per ml.

42. The method of any of claims 37-41, wherein the ECs are human umbilical vein endothelial cells (HUVECs).

43. The method of any of claims 37-42, wherein the freezing medium comprises human serum albumin (HSA).

44. The method of any of claims 37-43, wherein the freezing medium comprises about 10% human serum albumin (HSA).

45. The method of any of claims 37-44, wherein the cryopreservative is selected from the group consisting of dimethyl sulfoxide, ethylene glycol, propylene glycol, and glycerol.

46. The method of any of claims 37-45, wherein the cryopreservative is dimethyl sulfoxide.

47. The method of any of claims 37-46, wherein the freezing medium comprises from about 5% to about 10% dimethyl sulfoxide.

48. The method of any of claims 37-47, wherein the freezing medium comprises about 10% dimethyl sulfoxide.

49. The method of any of claims 37-48, wherein the freezing medium comprises a cell culture medium.

50. The method of any of claims 37-49, wherein the freezing medium comprises an endothelial growth medium.

51. The method of any of claims 37-50, wherein the endothelial cells (ECs) are adenovirus E4ORF1+ECs.

52. The method of any of claims 37-51, wherein the ECs comprise a recombinant nucleotide sequence that encodes an adenovirus E4ORF1 protein.

53. The method of claim 52, wherein the wherein the nucleotide sequence is operatively linked to a heterologous promoter.

54. The method of claim 52 or 53, wherein the nucleotide sequence is within a vector.

55. The method of claim 54, wherein the vector is a retroviral vector.

56. The method of claim 55, wherein the retroviral vector is a lentiviral vector.

57. The method of claim 55, wherein the retroviral vector is a Maloney murine leukemia virus (MMLV) vector.

58. The method of any of claims 51-57, wherein the E4ORF1 is human adenovirus type 5 E4ORF1.

59. The method of any of claims 51-58, wherein the ECs do not comprise an entire adenovirus E4 region.

60. The method of any of claims 51-59, wherein the ECs do not comprise do not comprise an E4ORF2, E4ORF3, E4ORF4, E4ORF5 or E4ORF6 coding sequence or amino acid sequence.

61. The method of any of claims 37-60, wherein the freezing composition further comprises hematopoietic stem cells or hematopoietic progenitor cells.

62. The method of claim 61, wherein the hematopoietic stem cells or hematopoietic progenitor cells are from bone marrow.

63. The method of claim 61, wherein the hematopoietic stem cells or hematopoietic progenitor cells are from peripheral blood.

64. The method of claim 61, wherein the hematopoietic stem cells or hematopoietic progenitor cells are from amniotic fluid.

65. The method of claim 61, wherein the hematopoietic stem cells or hematopoietic progenitor cells are from umbilical cord blood.

66. The method of any of claims 37-65, wherein the freezing composition is in a freezing container.

67. The method of claim 66, wherein the freezing container is a cryovial.

68. The method of claim 66, wherein the freezing container is a cryobag.

69. The method of claim 66, wherein the freezing container is adapted for aseptic transfer of its contents to a patient in a closed system.


SEQUENCE LISTING (1 sequence)

SEQ ID NO: 1 - protein, 128 aa, Unknown
  Description of Unknown Adenoviral sequence
MAAAVEALFVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV